(19) United States
(12) Patent Application Publication
(10) Pub. No.: US 2016/0152531 A1
(43) Pub. Date: Jun. 2, 2016

(54) METHOD FOR PRODUCING 1,3-BUTADIEN FROM N-BUTENES BY MEANS OF AN OXIDATIVE DEHYDROGENATION

(71) Applicant: BASF SE, Ludwigshafen (DE)

(72) Inventors: Christian WALSDORFF, Ludwigshafen (DE); Philipp GRÜNE, Mannheim (DE); Christine SCHMITT, Mannheim (DE); Ragavendra Prasad BALEGEDDE RAMACHANDRAN, Limburgerhof (DE); Jan Pablo JOSCH, Neustadt (DE)

(21) Appl. No.: 14/905,497

(22) PCT Filed: Jul. 15, 2014
(86) PCT No.: PCT/EP2014/065068

§ 371 Date: Jan. 15, 2016

(30) Foreign Application Priority Data
Jul. 18, 2013 (EP) 13177081.0

(51) Int. Cl. C07C 5/48; C07C 7/11

(52) U.S. Cl. CPC C07C 5/48; C07C 7/11

(57) ABSTRACT

The invention relates to a process for preparing butadiene from n-butenes, comprising the steps of:
A) providing an input gas stream a comprising n-butenes;
B) feeding the input gas stream a comprising n-butenes and an oxygenous gas into at least one oxidative dehydrogenation zone and oxidatively dehydrogenating n-butenes to butadiene, giving a product gas stream b comprising butadiene, unconverted n-butenes, water vapor, oxygen, low-boiling hydrocarbons and high-boiling secondary components, with or without carbon oxides and with or without inert gases;
Ca) cooling the product gas stream b by contacting it with a coolant and condensing at least a portion of the high-boiling secondary components;
Cb) compressing the remaining product gas stream b in at least one compression stage, giving at least one aqueous condensate stream c1 and one gas stream c2 comprising butadiene, n-butenes, water vapor, oxygen and low-boiling hydrocarbons, with or without carbon oxides and with or without inert gases;
Da) removing uncondensable and low-boiling gas constituents comprising oxygen and low-boiling hydrocarbons, with or without carbon oxides and with or without inert gases, as gas stream d2 from the gas stream c2 by absorbing the C₄ hydrocarbons comprising butadiene and n-butenes in an absorbent, giving an absorbent stream laden with C₄ hydrocarbons and the gas stream d2, and
Db) subsequently desorbing the C₄ hydrocarbons from the laden absorbent stream, giving a C₄ product gas stream d1,
which comprises additionally feeding in a methane-comprising gas stream at at least one point in the process section comprising steps B), Ca), Cb) and Da) in such amounts that the formation of an explosive gas mixture in step Da) is avoided.

The invention relates to a process for preparing 1,3-butadiene from n-butenes by oxidative dehydrogenation (OOH).

Butadiene is an important base chemical and is used, for example, for production of synthetic rubbers (butadiene homopolymers, styrene-butadiene rubber or nitrile rubber) or for production of thermoplastic terpolymers (acrylonitrile-butadiene-styrene copolymers). Butadiene is also converted to sulfolane, chloroprene and 1,4-hexamethylenediamine (via 1,4-dichlorobutene and adiponitrile). Through dimerization of butadiene, it is also possible to obtain vinylcyclohexene, which can be dehydrogenated to styrene.

Butadiene can be prepared by thermal cracking (steamcracking) of saturated hydrocarbons, typically proceeding from naphtha as the raw material. The steamcracking of naphtha affords a hydrocarbon mixture of methane, ethane, ethene, acetylene, propane, propene, propyne, allene, butanes, butenes, butadiene, butynes, methylallene, and C₅ and higher hydrocarbons.

Butadiene can also be obtained by the oxidative dehydrogenation of n-butenes (1-butene and/or 2-butene). The starting gas mixture utilized for the oxidative dehydrogenation (oxydehydrogenation, ODH) of n-butenes to butadiene may be any desired mixture comprising n-butenes. For example, it is possible to use a fraction which comprises n-butenes (1-butene and/or 2-butene) as the main constituent and has been obtained from the C₄ fraction from a naphtha cracker by removing butadiene and isobutene. In addition, it is also possible to use gas mixtures which comprise 1-butene, cis-2-butene, trans-2-butene or mixtures thereof and have been obtained by dimerization of ethylene as the starting gas. In addition, the starting gases used may be gas mixtures which comprise n-butenes and have been obtained by catalytic fluidized bed cracking (fluid catalytic cracking, FCC).

Processes for oxidative dehydrogenation of butenes to butadiene are known in principle.

US 2012/0130137A1, for example, describes a process of this kind using catalysts comprising oxides of molybdenum, bismuth and generally further metals. For the lasting activity of such catalysts for the oxidative dehydrogenation, a critical minimum level of partial oxygen pressure is required in the gas atmosphere in order to avoid an excessive reduction and hence loss of performance of the catalysts. For this reason, it is generally also not possible to work with a stoichiometric oxygen input or complete oxygen conversion in the oxydehydrogenation reactor (ODH reactor). US 2012/0130137 A1 describes, for example, an oxygen content of 2.5-8% by volume in the starting gas.

A subject of particular discussion in paragraph [0017] is the problem of formation of possible explosive mixtures after the reaction step. More particularly, it is pointed out that, in what is called a “rich” mode of operation, above the upper explosion limit in the reaction section, there exists the problem that, after absorption of a majority of the organic constituents in the workup, the gas composition crosses the explosive range in the course of a transition from rich to lean gas mixture. On the other hand, it is also pointed out that, in the case of a constantly lean mode of operation in the reaction section, there exists the risk of catalyst deactivation through coking. US 2012/0130137 A1, however, does not mention any solution to this problem. Paragraph [106] mentions incidentally how the occurrence of explosive atmospheres in the absorption step can be avoided, for example, by a dilution of the gas stream with nitrogen prior to the absorption step. The more detailed description of the absorption step in paragraphs [0132] ff. does not address the problem of formation of explosive gas mixtures any further.

The need for an oxygen excess for such catalyst systems is common knowledge and is reflected in the process conditions when catalysts of this kind are used. Representative examples include the comparatively recent studies by Jung et al. (Catal. Surv. Asia 2009, 13, 78-93; DOI 10.1007/s10563-009-9069-5 and Applied Catalysis A: General 2007, 317, 244-249; DOI 10.1016/j.apcata.2006.10.021).

The occurrence of high oxygen concentrations alongside hydrocarbons such as butane, butene and butadiene or the organic absorbents used in the workup section, however, is afflicted with risks. For instance, explosive gas mixtures can form. If only a small margin from the explosive range is employed, it is not always possible in industry to prevent this range from being entered through variations in the process parameters.

U.S. Pat. No. 4,504,692 (Japan Synthetic Rubber Co. Ltd.) describes a process for preparing butadiene, especially with removal of isobutene. However, there is no mention of the problem of the formation or of the avoidance of explosive gas mixtures.

EP 2 177 266 A2 describes a catalyst based on Bi/Mo/Fe oxides and a corresponding process for the oxidative dehydrogenation of butenes to butadiene. However, there is no mention of the problem of the formation or of the avoidance of explosive gas mixtures.

KR 2013036467 A describes a process for oxidative dehydrogenation of butene to butadiene, especially with avoidance of fouling in the downstream region. However, there is no mention of the problem of the formation or of the avoidance of explosive gas mixtures.

U.S. Pat. No. 7,034,195 B2 describes suitable catalysts for the oxidative dehydrogenation of butenes to butadiene. Also described is the presence of low boilers such as methane in amounts of <5% by volume. However, the problem of the formation or of the avoidance of explosive mixtures is not discussed.

U.S. Pat. No. 8,003,840 B2 likewise describes a catalyst based on Bi/Mo/Fe oxides and a corresponding process for the oxidative dehydrogenation of butenes to butadiene. However, the problem of the formation or of the avoidance of explosive mixtures is not discussed.

One disadvantage of the prior art processes is thus the passage through a region of explosive gas mixtures in the course of workup of the oxygenous product gas mixture when the composition of the oxygenous product gas mixture comprising C₄ hydrocarbons changes from a (non-explosive) “rich” to a (non-explosive) “lean” mixture in the course of absorption of the C₄ hydrocarbons.

The present invention eliminates the disadvantages mentioned by feeding a separate methane-comprising gas stream into the process at one or more points, such that a non-explosive hydrocarbon-rich “rich” gas mixture is always present in the course of workup of the oxygenous product gas mixture comprising C₄ hydrocarbons. More particularly, some or all of the nitrogen used as a diluent gas or present in air is replaced by methane.

The object is thus achieved by a process for preparing butadiene from n-butenes, comprising the steps of:

A) providing an input gas stream a comprising n-butenes;
B) feeding the input gas stream a comprising n-butenes and an oxygenous gas into at least one oxidative dehydrogenation zone and oxidatively dehydrogenating n-butenes to butadiene, giving a product gas stream b comprising butadiene, unconverted n-butenes, water vapor, oxygen, low-boiling hydrocarbons and high-boiling secondary components, with or without carbon oxides and with or without inert gases;
Ca) cooling the product gas stream b by contacting it with a coolant and condensing at least a portion of the high-boiling secondary components;
Cb) compressing the remaining product gas stream b in at least one compression stage, giving at least one aqueous condensate stream c1 and one gas stream c2 comprising butadiene, n-butenes, water vapor, oxygen and low-boiling hydrocarbons, with or without carbon oxides and with or without inert gases;
Da) removing uncondensable and low-boiling gas constituents comprising oxygen and low-boiling hydrocarbons, with or without carbon oxides and with or without inert gases, as gas stream d2 from the gas stream c2 by absorbing the C₄ hydrocarbons comprising butadiene and n-butenes in an absorbent, giving an absorbent stream laden with C₄ hydrocarbons and the gas stream d2, and
Db) subsequently desorbing the C₄ hydrocarbons from the laden absorbent stream, giving a C4 product gas stream d1, which comprises additionally feeding in a methane-comprising gas stream at at least one point in the process section comprising steps B), Ca), Cb) and Da) in such amounts that the formation of an explosive gas mixture in step Da) is avoided.

Preferably, steps E) and F) are also performed subsequently:

E) separating the C₄ product stream d1 by extractive distillation with a butadiene-selective solvent into a stream e1 comprising butadiene and the selective solvent and a stream e2 comprising n-butenes;
F) distilling the stream f2 comprising butadiene and the selective solvent into a stream g1 consisting essentially of the selective solvent and a stream g2 comprising butadiene.

In a preferred variant, the methane-comprising gas stream is fed into step B). In a further preferred variant, the methane-comprising gas stream is fed into step Da). The methane-comprising gas stream may also be fed in between steps B) and Da). It is also possible to feed in a plurality of methane-comprising gas streams at different points in the process.

The feeding-in of one or more separate methane-comprising gas streams at one or more points in the process section comprising steps B), Ca), Cb) and Da), i.e. feeding into these steps or between these steps, ensures that a sufficiently “rich”, i.e. hydrocarbon-rich, gas mixture is always present in the removal step Da) to reliably avoid the formation of an explosive gas mixture during the absorption step. Preferably, a margin of at least 2% by volume of oxygen is maintained from the explosive range. The explosive range is for specific to the components present in the mixture and can be found in databases, or be determined via experiments with the mixture in different compositions. These experimental methods are known to those skilled in the art.

In general, a sufficient amount of methane is fed in that the methane content of the gas stream d2 removed in step Da) is at least 15% by volume. Preference is given to feeding in a sufficient amount of methane that the methane content of the gas stream d2 obtained in step Da) is at least 20% by volume.

In general, the methane-comprising gas stream d2 removed in step Da) is at least partly recycled into step B). The proportion of the recycled portion in the overall stream is guided by the proportion of nitrogen which is replaced by methane.

In one embodiment, preference is given to using an oxygenous gas comprising more than 10% by volume, preferably more than 15% by volume and more preferably more than 20% by volume of molecular oxygen. In one embodiment, air is used as the oxygenous gas. In that case, the upper limit for the content of molecular oxygen in the oxygenous gas is generally 50% by volume or less, preferably 30% by volume or less and even more preferably 25% by volume or less. In addition, any desired inert gases may be present gas in the molecular oxygen-comprising gas. Possible inert gases may include nitrogen, argon, neon, helium, CO, CO₂ and water. The amount of inert gases in the oxygenous gas, for nitrogen, is generally 90% by volume or less, preferably 85% by volume or less and even more preferably 80% by volume or less. In the case of constituents other than nitrogen in the oxygenous gas, it is generally 10% by volume or less, preferably 1% by volume or less.

In a further embodiment, it is preferable to replace a maximum amount of nitrogen with methane and to recycle stream d2 essentially completely. In that case, it is especially preferable to use oxygen of maximum purity as the oxygenous gas.

In one embodiment, methane is thus used in place of nitrogen as the diluent gas. In that case, it is preferable to feed as little nitrogen as possible into the process. In that case, the nitrogen content of the gas stream d2 obtained in step Da) is preferably at most 10% by volume. In that case, a gas stream of maximum oxygen content is preferably fed into step B) of the process. Preference is given to feeding in an oxygen-containing gas having an oxygen content of at least 90% by volume. Particular preference is given to feeding in oxygen of technical-grade purity having a content of at least 95% oxygen.

By means of scrubbing, it is possible to reduce the COx content in the gas stream d2 before the gas stream d2 is recycled into stage B). Such a scrubbing operation is especially preferred when all of the nitrogen is replaced by methane and the majority of the gas stream d2 is recycled. Preference is given to scrubbing in scrubbers operated with amines for removal of CO₂.

In one embodiment of the invention, the gas stream d2 present in step Da) is recycled into step B) to an extent of at least 80%, preferably to an extent of at least 90%. This may be advisable when only a small purge stream has to be discharged from the gas stream d2.

In general, an aqueous coolant or an organic solvent is used in the cooling stage Co).

Preference is given to using an organic solvent in the cooling stage Ca). These organic solvents generally have a very much higher dissolution capacity for the high-boiling by-products which can lead to deposits and blockages in the plant parts downstream of the ODH reactor than water or aqueous alkaline solutions. Organic solvents used with preference as coolants are aromatic hydrocarbons, for example toluene, o-xylene, m-xylene, p-xylene, diethylbenzenes, triethylbenzenes, diisopropylbenzenes, triisopropylbenzenes and mesitylene or mixtures thereof. Particular preference is given to mesitylene.

Embodiments which follow are preferred or particularly preferred variants of the process according to the invention: Stage Ca) is performed in multiple stages in stages Ca1) to Can), preferably in two stages Ca1) and Ca2). In this case, particular preference is given to feeding at least a portion of the solvent as a coolant to the first stage Ca1) after it has passed through the second stage Ca2).

Stage Cb) generally comprises at least one compression stage Cba) and at least one cooling stage Cbb). Preferably, in the at least one cooling stage Cbb), the gas compressed in the compression stage Cba) is contacted with a coolant. More preferably, the coolant in the cooling stage Cbb) comprises the same organic solvent which is used as a coolant in stage Ca). In an especially preferred variant, at least some of this coolant is fed as a coolant to stage Ca) after it has passed through the at least one cooling stage Cbb).

Preferably, stage Cb) comprises a plurality of compression stages Cba1) to Cban) and cooling stages Cbb1) to Cbbn), for example four compression stages Cba1) to Cba4) and four cooling stages Cbb1) to Cbb4).

Preferably, step D) comprises steps Da1), Da2) and Db):

Da1) absorbing the C₄ hydrocarbons comprising butadiene and n-butenes in a high-boiling absorbent, giving an absorbent stream laden with C₄ hydrocarbons and the gas stream d2,

Da2) removing oxygen from the absorbent stream laden with C₄ hydrocarbons from step Da) by stripping with an uncondensable gas stream, and

Db) desorbing the C₄ hydrocarbons from the laden absorbent stream, giving a C₄ product gas stream d1 consisting essentially of C₄ hydrocarbons and comprising less than 100 ppm of oxygen.

Preferably, the high-boiling absorbent used in step Da) is an aromatic hydrocarbon solvent, more preferably the aromatic hydrocarbon solvent used in step Ca), especially mesitylene. It is also possible to use diethylbenzenes, triethylbenzenes, diisopropylbenzenes and triisopropylbenzenes.

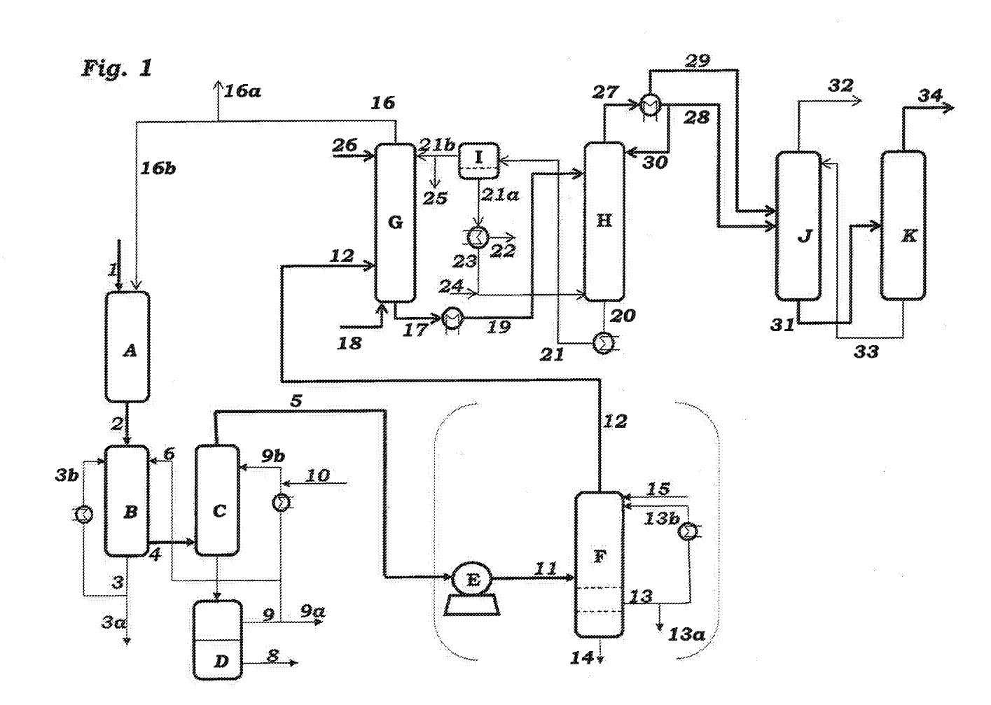

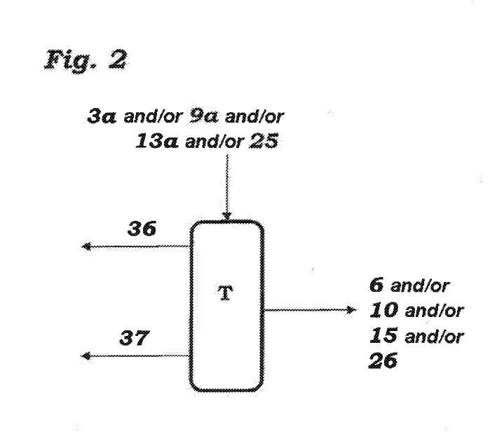

Embodiments of the process according to the invention are presented in FIGS. 1 and 2 and are described in detail hereinafter.

The input gas streams used may be pure n-butenes (1-bütene and/or cis-/trans-2-butene), but also gas mixtures comprising butenes. Such a gas mixture can be obtained, for example, by nonoxidative dehydrogenation of n-butane. It is also possible to use a fraction which comprises n-butenes (1-butene and cis-/trans-2-butene) as the main constituent and has been obtained from the C₄ fraction from naphtha cracking by removal of butadiene and isobutene. In addition, the starting gases used may also be gas mixtures which comprise pure 1-butene, cis-2-butene, trans-2-butene or mixtures thereof, and which have been obtained by dimerization of ethylene. In addition, the starting gases used may be gas mixtures comprising n-butenes, which have been obtained by catalytic fluidized bed cracking (fluid catalytic cracking, FCC).

In one embodiment of the process according to the invention, the starting gas mixture comprising n-butenes is obtained by nonoxidative dehydrogenation of n-butane. Through the coupling of a nonoxidative catalytic dehydrogenation with the oxidative dehydrogenation of the n-butenes formed, it is possible to obtain a high yield of butadiene, based on n-butane used, The nonoxidative catalytic n-butane dehydrogenation gives a gas mixture which, as well as butadiene, comprises 1-butene, 2-butene and unconverted n-butane secondary constituents. Typical secondary constituents are hydrogen, water vapor, nitrogen, CO and CO₂, methane, ethane, ethene, propane and propene. The composition of the gas mixture leaving the first dehydrogenation zone may vary significantly depending on the mode of operation of the dehydrogenation. For instance, in the case of performance of the dehydrogenation while feeding in oxygen and additional hydrogen, the product gas mixture has a comparatively high content of water vapor and carbon oxides. In the case of modes of operation without feeding of oxygen, the product gas mixture of the nonoxidative dehydrogenation has a comparatively high content of hydrogen.

In step B) the input gas stream comprising n-butenes and an oxygenous gas are fed into at least one dehydrogenation zone (the ODH reactor A) and the butenes present in the gas mixture are oxidatively dehydrogenated to butadiene in the presence of an oxydehydrogenation catalyst.

The methane content of the gas mixture converted in step B), when a methane-comprising gas stream is fed into step B), is generally at least 10% by volume, preferably at least 20% by volume. It is generally at most 90% by volume.

A methane-comprising gas stream can be fed into step B) as a fresh methane gas stream and/or as a methane-comprising recycle stream d2.

Catalysts suitable for the oxydehydrogenation are generally based on an Mo—Bi—O-containing multimetal oxide system which generally additionally comprises iron. In general, the catalyst system also comprises further additional components, for example potassium, cesium, magnesium, zirconium, chromium, nickel, cobalt, cadmium, tin, lead, germanium, lanthanum, manganese, tungsten, phosphorus, cerium, aluminum or silicon. Iron-containing ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

Examples of Mo—Bi—Fe—O-containing multimetal oxides are Mo—Bi—Fe—Cr—O— or Mo—Bi—Fe—Zr—O-containing multimetal oxides. Preferred systems are described, for example, in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}La_{0.5}K_{0.1}Oₓ+SiO₂), DE-A 25 30 959 (Mo₁₂BiFe₃Co_{4.5}Ni_{2.5}Cr_{0.5}K_{0.1}Oₓ, Mo_{13.75}BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo₁₂BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and the preparation thereof are additionally described in U.S. Pat. No. 4,423,281 (Mo₁₂BiNi₈Pb_{0.5}Cr₃K_{0.2}Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}Oₓ SiO₂) and DE-A 24 40 329 (Mo₁₂BiCo4.5Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia)

where
X¹=Si, Mn and/or Al,
X²=Li, Na, K, Cs and/or Rb,
0.2≦a≦1,
0.5≦b≦10,
0≦c≦10,
0≦d≦10,
2≦c+d≦10,
0≦e≦2,
0≦f≦10,
0≦g≦0.5,
y=a number which is determined with the prerequisite of charge neutrality by the valency and frequency of the elements in (la) other than oxygen.

Preference is given to catalysts whose catalytically active oxide composition, of the two metals Co and Ni, has only Co (d=0). Preferred is X¹ Si and/or Mn and X² is preferably K, Na and/or Cs, more preferably X²=K. Particular preference is given to a substantially Cr(VI)-free catalyst.

For performance of the oxidative dehydrogenation at high overall conversion of n-butenes, preference is given to a gas mixture having a molar oxygen:n-butenes ratio of at least 0.5. Preference is given to working at an oxygen:n-butenes ratio of 0.55 to 10. To set this value, the starting material gas can be mixed with oxygen or an oxygenous gas and optionally additional inert gas, methane or water vapor. The oxygenous gas mixture obtained is then fed to the oxydehydrogenation.

The reaction temperature in the oxydehydrogenation is generally controlled by a heat exchange medium present around the reaction tubes. Examples of useful liquid heat exchange media of this kind include melts of salts or salt mixtures such as potassium nitrate, potassium nitrite, sodium nitrite and/or sodium nitrate, and melts of metals such as sodium, mercury and alloys of various metals. It is also possible to use ionic liquids or heat carrier oils. The temperature of the heat exchange medium is between 220 to 490° C. and preferably between 300 to 450° C. and more preferably between 350 and 420° C.

Because of the exothermicity of the reactions which proceed, the temperature in particular sections of the reaction interior during the reaction may be higher than that of the heat exchange medium, and what is called a hotspot develops. The position and magnitude of the hotspot is decided by the reaction conditions, but it can also be regulated through the dilution ratio of the catalyst layer or the flow rate of mixed gas. The difference between hotspot temperature and the temperature of the heat exchange medium is generally between 1-150° C., preferably between 10-100° C. and more preferably between 20-80° C. The temperature at the end of the catalyst bed is generally between 0-100° C., preferably between 0.1-50° C., more preferably between 1-25° C., above the temperature of the heat exchange medium.

The oxydehydrogenation can be performed in all fixed bed reactors known from the prior art, for example in a staged oven, in a fixed bed tubular reactor or shell and tube reactor, or in a plate heat exchanger reactor. A shell and tube reactor is preferred.

Preferably, the oxidative dehydrogenation is performed in fixed bed tubular reactors or fixed bed shell and tube reactors. The reaction tubes (just like the other elements of the shell and tube reactor) are generally manufactured from steel. The wall thickness of the reaction tubes is typically 1 to 3 mm. The internal diameter thereof is generally (homogeneously) 10 to 50 mm or 15 to 40 mm, frequently 20 to 30 mm. The number of reaction tubes accommodated in a shell and tube reactor is generally at least 1000, or 3000, or 5000, preferably at least 10 000. Frequently, the number of reaction tubes accommodated in a shell and tube reactor is 15 000 to 30 000 or to 40 000 or to 50 000. The length of the reaction tubes normally extends to a few meters, atypical reaction tube length being in the range from 1 to 8 m, frequently 2 to 7 m, in many cases 2.5 to 6 m.

In addition, the catalyst layer set up in the ODH reactor A may consist of a single layer or of 2 or more layers. These layers may consist of pure catalyst or be diluted with a material which does not react with the starting gas or components from the product gas of the reaction. In addition, the catalyst layers may consist of unsupported material and/or supported eggshell catalysts. The product gas stream 2 leaving the oxidative dehydrogenation comprises, as well as butadiene, generally also unconverted 1-butene and 2-butene, oxygen and water vapor. As secondary components, it generally further comprises carbon monoxide, carbon dioxide, inert gases (principally nitrogen), low-boiling hydrocarbons such as methane, ethane, ethene, propane and propene, butane and isobutane, with or without hydrogen and with or without oxygen-containing hydrocarbons, called oxygenates. Oxygenates may, for example, be formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

Product gas stream 2 at the reactor outlet is characterized by a temperature close to the temperature at the end of the catalyst bed. The product gas stream is then brought to a temperature of 150-400° C., preferably 160-300° C., more preferably 170-250° C. In order to keep the temperature within the desired range, it is possible to insulate the line through which the product gas stream flows or to use a heat exchanger. This heat exchanger system is as desired, provided that this system can be used to keep the temperature of the product gas at the desired level. Examples of a heat exchanger include spiral heat exchangers, plate heat exchangers, double tube heat exchangers, multitube heat exchangers, boiler-spiral heat exchangers, boiler-shell heat exchangers, liquid-liquid contact heat exchangers, air heat exchangers, direct contact heat exchangers and fin tube heat exchangers. Since, while the temperature of the product gas is set to the desired temperature, some of the high-boiling by-products present in the product gas can precipitate out, the heat exchanger system should preferably have two or more heat exchangers. If two or more heat exchangers provided are arranged in parallel in this case, and distributed cooling of the product gas obtained in the heat exchangers is thus enabled, the amount of high-boiling by-products which are deposited in the heat exchangers decreases, and hence the service life thereof can be extended. As an alternative to the abovementioned method, the two or more heat exchangers provided may be arranged in parallel. The product gas is supplied to one or more, but not to all, heat exchangers, which are succeeded by other heat exchangers after a certain operation period. In the case of this method, the cooling can be continued, some of the heat of reaction can be recovered and, in parallel, the high-boiling by-products deposited in one of the heat exchangers can be removed. It is possible to use a solvent as an abovementioned coolant, provided that it is capable of dissolving the high-boiling by-products. Examples are aromatic hydrocarbon solvents, for example toluene and xylenes, diethylbenzenes, triethylbenzenes, diisopropylbenzenes and triisopropylbenzenes. Particular preference is given to mesitylene. It is also possible to use aqueous solvents. These may be either acidified or alkalized, for example an aqueous solution of sodium hydroxide.

Subsequently, a majority of the high-boiling secondary components and of the water is removed from product gas stream 2 by cooling and compression. The cooling is effected by contacting with a coolant. This stage is also referred to hereinafter as the quench. This quench may consist of only one stage or of several stages (for example B. C in FIG. 1). Product gas stream 2 is thus contacted directly with the organic cooling medium 3b and 9b and hence cooled. Suitable cooling media are aqueous coolants or organic solvents, preferably aromatic hydrocarbons, more preferably toluene, o-xylene, m-xylene, p-xylene or mesitylene, or mixtures thereof, used. It is also possible to use diethylbenzene, triethylbenzene, diisopropylbenzene and triisopropylbenzene.

Preference is given to a two-stage quench (comprising stages B and C according to FIG. 1), i.e. stage Ca) comprises two cooling stages Ca1) and Ca2) in which product gas stream 2 is contacted with the organic solvent.

In general, product gas 2, according to the presence and temperature level of a heat exchanger upstream of the quench B, has a temperature of 100-440° C. The product gas is contacted in the 1st quench stage B with the cooling medium composed of organic solvent. It is possible here to introduce the cooling medium through a nozzle in order to achieve very efficient mixing with the product gas. For the same purpose, it is possible to introduce internals, for example further nozzles, in the quench stage, through which the product gas and the cooling medium pass together. The coolant inlet into the quench is designed such that blockage by deposits in the region of the coolant inlet is minimized.

In general, product gas 2 is cooled in the first quench stage B to 5-180° C., preferably to 30-130° C. and even more preferably to 60-110° C. The temperature of the coolant medium 3b at the inlet may generally be 25-200° C., preferably 40-120° C., especially preferably 50-90° C. The pressure in the first quench stage B is not particularly restricted, but is generally 0.01-4 bar (a), preferably 0.1-2 bar (a) and more preferably 0.2-1 bar (a). When greater amounts of high-boiling by-products are present in the product gas, high-boiling by-products may readily polymerize and result in deposits of solids which are caused by high-boiling by-products in this process section.

In general, the quench stage B is configured as a cooling tower. The cooling medium 3b used in the cooling tower is frequently used in circulating form. The circulation flow rate of the cooling medium in liters per hour, based on the mass flow rate of butadiene in grams per hour, may generally be 0.0001-51/g, preferably 0.001-1I/g and more preferably 0.002-0.2 l/g.

The temperature of the cooling medium 3 in the bottom may generally be 27-210° C., preferably 45-130° C., especially preferably 55-95° C. Since the loading of the cooling medium 4 with secondary components increases over the course of time, a portion of the laden cooling medium can be drawn off as a purge stream 3a from the circulation and the circulation rate can be kept constant by adding unladen cooling medium 6. The ratio of outflow rate and addition rate depends on the steam loading of the product gas and the product gas temperature at the end of the first quench stage.

The cooled product gas stream 4 which has possibly been depleted of secondary components can then be sent to a second quench stage C. It can then be contacted again with a cooling medium 9b therein.

In general, the product gas up to the gas outlet of the second quench stage C can be cooled to 5 to 100° C., preferably to 15-85° C. and even more preferably to 30-70° C. The coolant can be fed in countercurrent to the product gas. In this case, the temperature of the coolant medium 9b at the coolant inlet may be 5-100° C., preferably 15-85° C., especially preferably 30-70° C. The pressure in the second quench stage C is not particularly restricted, but is generally 0.01-4 bar (a), preferably 0.1-2 bar (a) and more preferably 0.2-1 bar (a). The second quench stage 7 is preferably configured as a cooling tower. The cooling medium 9b used in the cooling tower is frequently used in circulating form. The circulation flow rate of the cooling medium 9b in liters per hour, based on the mass flow rate of butadiene in grams per hour, may generally be 0.0001-5 l/g, preferably 0.3001-1 l/g and more preferably 0.002-0.21/g.

According to the temperature, pressure, coolant and water content of the product gas 4, there may be condensation of water in the second quench stage C. In this case, an additional aqueous phase 8 may form, which may additionally comprise water-soluble secondary components. In that case, these can be drawn off in the phase separator D. The temperature of the cooling medium 9 in the bottom may generally be 20-210° C., preferably 35-120° C., especially preferably 45-85° C. Since the loading of the cooling medium 9 with secondary components increases over the course of time, a portion of the laden cooling medium can be drawn off as a purge stream 9a from the circulation and the circulation rate can be kept constant by adding unladen cooling medium 10.

In order to achieve very good contact of product gas and cooling medium, internals may be present in the second quench stage C. Such internals comprise, for example, bubble-cap trays, centrifugal trays and/or sieve trays, columns having structured packings, for example sheet metal packings having a specific surface area of 100 to 1000 m²/m³, such as Mellapak® 250 Y, and columns having random packings.

The solvent circulation streams of the two quench stages may either be separate from one another or combined with one another. For example, stream 9a may be fed into stream 3b or replace it. The desired temperature of the circulation streams can be set by means of suitable heat exchangers.

In a preferred embodiment of the invention, the cooling stage Ca) is performed in two stages, in which case the solvent laden with secondary components from the second stage Ca2) is conducted into the first stage Ca1). The solvent withdrawn from the second stage Ca2) comprises a lower level of secondary components than the solvent withdrawn from the first stage Ca1).

In order to minimize the entrainment of liquid constituents from the quench into the offgas line, suitable construction measures, for example the installation of a demister, can be taken. In addition, high-boiling substances which are not separated from the product gas in the quench can be removed from the product gas through further construction measures, for example further gas scrubbing operations.

A as stream 5 is obtained, comprising n-butane, 1-butene, 2-butenes and butadiene, with or without oxygen, hydrogen and water vapor, and small amounts of methane, ethane, ethene, propane and propene, isobutane, carbon oxides, inert gases and portions of the solvent used in the quench. In addition, traces of high-boiling components which have not been removed quantitatively in the quench may remain in this gas stream 5.

Subsequently, the gas stream b from the cooling step Ca), which has been depleted of high-boiling secondary components, is cooled in step Cb) in at least one compression stage Cba) and preferably in at least one cooling stage Cbb) by contacting with an organic solvent as a coolant.

The product gas stream 5 from the solvent quench is compressed in at least one compression stage E and subsequently cooled further in the cooling apparatus F, forming at least one condensate stream 14. There remains a gas stream 12 comprising butadiene, 1-butene, 2-butenes, oxygen and water vapor, with or without low-boiling hydrocarbons such as methane, ethane, ethene, propane and propene, butane and isobutane, with or without carbon oxides and with or without inert gases. In addition, this product gas stream may also comprise traces of high-boiling components.

The compression and cooling of the gas stream 5 can be effected in one or more stages (n stages). In general, compression is effected overall from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream may thus also comprise several streams in the case of multistage compression. The condensate stream consists of large portions of water and the solvent used in the quench. Both streams (aqueous and organic phase) may additionally comprise, to a small extent, secondary components such as low boilers, C₄ hydrocarbons, oxygenates and carbon oxides.

In order to cool the stream 11 which arises through compression of stream 5 and/or in order to remove further secondary components from the stream 11, the condensed quench solvent can be cooled in a heat exchanger and recycled as coolant into the apparatus F. Since the loading of this cooling medium 13b with secondary components increases over the course of time, a portion of the laden cooling medium can be drawn off from the circulation (13a) and the circulation rate of the cooling medium can be kept constant by adding unladen solvent (15).

The solvent 13b, which is added as the cooling medium, may be an aqueous coolant or an organic solvent. Preference is given to aromatic hydrocarbons, particular preference to toluene, o-xylene, m-xylene, p-xylene, diethylbenzene, triethylbenzene, diisopropylbenzene, triisopropylbenzene, mesitylene or mixtures thereof. Particular preference is given to mesitylene.

The condensate stream 13a can be recycled into the circulation stream 3b and/or 9b of the quench. As a result, the C₄ components absorbed in the condensate stream 13a can be brought back into the gas stream, and hence the yield can be increased. Suitable compressors are, for example, turbo compressors, rotary piston compressors and reciprocating piston compressors. The compressors may be driven, for example, with an electric motor, an expander or a gas or steam turbine. Typical compression ratios (exit pressure:inlet pressure) per compressor stage are, according to the design, between 1.5 and 3.0. The compressed gas is cooled in with coolant-flushed heat exchangers or organic quench stages which may be designed, for example, as shell and tube, spiral or plate heat exchangers. Suitable coolants may be aqueous solvents or the abovementioned organic solvents. The coolants used in the heat exchangers are cooling water or heat carrier oils. In addition, preference is given to using air cooling with use of blowers.

The gas stream 12 comprising butadiene, n-butenes, oxygen, low-boiling hydrocarbons (methane, ethane, ethene, propane, propene, n-butane, isobutane), with or, without water vapor, with or without carbon oxides and with or without inert gases and with or without traces of secondary components is fed as an output stream to further processing. According to the invention, methane can additionally be added to the gas stream 12, in order to ensure that a non-explosive, hydrocarbon-rich “rich” gas mixture is always present in the column G.

In a step D), uncondensable and low-boiling gas constituents comprising oxygen, low-boiling hydrocarbons (methane, ethane, ethene, propane, propene), carbon oxides and inert gases are separated in an absorption column G as gas stream 16 from the process gas stream 12 by absorption of the C₄ hydrocarbons in a high-boiling absorbent (20b and/or 23) and subsequent desorption of the C₄ hydrocarbons. Preferably, step D), as shown in FIG. 1, comprises steps Da1), Da2) and Db):
Da1) absorbing the C₄ hydrocarbons comprising butadiene and n-butenes in a high-boiling absorbent (21b and/or 26), giving an absorbent stream laden with C₄ hydrocarbons and the gas stream 16,
Da2) removing oxygen from the absorbent stream laden with C₄ hydrocarbons from step Da) by stripping with an uncondensable gas stream 18, giving an absorbent stream 17 laden with C₄ hydrocarbons, and
Db) desorbing the C₄ hydrocarbons from the laden absorbent stream, giving a C₄ product gas stream 27 consisting essentially of C₄ hydrocarbons.

For this purpose, in the absorption stage G, the gas stream 12 is contacted with an inert absorbent and the C₄ hydrocarbons are absorbed in the inert absorbent, giving an absorbent laden with C₄ hydrocarbons and an offgas 16 comprising the other gas constituents. In a desorption stage, the C₄ hydrocarbons are released again from the high-boiling absorbent.

It is essential to the invention that, during the absorption stage G and at every point in the absorption column, a non-explosive, hydrocarbon-rich rich gas mixture is always present. In general, the oxygen content of the gas mixture during the absorption stage G is in the range from 3 to 10% by volume. The hydrocarbon content (C₄ hydrocarbons+methane+low-boiling secondary constituents+vaporous absorbent) is generally in the range from 15 to 97% by volume.

The absorption stage can be performed in any desired suitable absorption column known to those skilled in the art. The absorption can be effected by simply passing the product gas stream through the absorbent. However, it can also be effected in columns or in rotary absorbers. It is possible to work in cocurrent, countercurrent or crosscurrent. The absorption is preferably conducted in countercurrent. Suitable absorption columns are, for example, tray columns having bubble-cap trays, centrifugal trays and/or sieve trays, columns having structured packings, for example sheet metal packings having a specific surface area of 100 to 1000 m²/m³ such as Mellapak® 250 Y, and columns having random packings. However, also useful are trickle towers and spray towers, graphite block absorbers, surface absorbers such as thick-layer and thin-layer absorbers, and also rotary columns, pan scrubbers, cross-spray scrubbers and rotary scrubbers.

In one embodiment, the gas stream 12 comprising butadiene, n-butenes and the low-boiling and uncondensable gas constituents is supplied to an absorption column in the lower region. In the upper region of the absorption column, the high-boiling absorbent (21b and/or 26) is introduced.

Inert absorbents used in the absorption stage are generally high-boiling nonpolar solvents in which the C₄ hydrocarbon mixture to be removed has a distinctly higher solubility than the other gas constituents to be removed. Suitable absorbents are comparatively nonpolar organic solvents, for example aliphatic C₈- to C₁₈-alkanes, or aromatic hydrocarbons such as the middle oil fractions from paraffin distillation, toluene or ethers having bulky groups, or mixtures of these solvents, to which a polar solvent such as dimethyl 1,2-phthalate may be added. Suitable absorbents are additionally esters of benzoic acid and phthalic acid with straight-chain C₁-C₈-alkanols, and what are called heat carrier oils, such as biphenyl and diphenyl ethers, chlorine derivatives thereof and triarylalkenes. A suitable absorbent is a mixture of biphenyl and diphenyl ether, preferably in the azeotropic composition, for example the commercially available Diphyl®. Frequently, this solvent mixture comprises dimethyl phthalate in an amount of 0.1 to 25% by weight.

In a preferred embodiment, the same solvent is used in the absorption stage Da1) as in the cooling stage Ca).

Preferred absorbents are solvents having a dissolution capacity for organic peroxides of at least 1000 ppm (mg of active oxygen/kg of solvent). Preference is given to aromatic hydrocarbons, particular preference to toluene, o-xylene, p-xylene and mesitylene, or mixtures thereof. It is also possible to use diethylbenzene, triethylbenzene, diisopropylbenzene and triisopropylbenzene.

At the top of the absorption column G, a stream 16 is drawn off, comprising essentially oxygen and low-boiling hydrocarbons (methane, ethane, ethene, propane, propene), with or without C₄ hydrocarbons (butane, butenes, butadiene), with or without inert gases, with or without carbon oxides and with or without water vapor. This stream can be supplied partly to the ODH reactor. It is thus possible, for example, to adjust the inlet stream of the ODH reactor to the desired C₄ hydrocarbon content. Moreover, it is possible in accordance with the invention, through this recycling operation, to return a large portion of the methane. In that case, the methane used is not completely incinerated but is at least partly reused.

The methane content of stream 16 is generally at least 15% by volume, preferably at least 20% by volume. In general, the methane content is at most 95% by volume. Stream 16 is divided and fed as stream 16b into the reactor A. The remaining substream 16a can be used thermally or physically, for example in a synthesis gas plant.

At the bottom of the absorption column, in a further column, purging with a gas 18 discharges residues of oxygen dissolved in the absorbent. The remaining oxygen content should be sufficiently small that the stream 27 which comprises butane, butene and butadiene and leaves the desorption column comprises only a maximum of 100 ppm of oxygen.

The stripping of the oxygen in step Db) can be performed in any desired suitable column known to those skilled in the art. The stripping can be effected by simply passing uncondensable gases, preferably gases absorbable only slightly, if at all, in the absorbent stream 21b and/or 26, such as methane, through the laden absorption solution. C₄ hydrocarbons additionally stripped out are washed back into the absorption solution in the upper portion of column G, by passing the gas stream back into this absorption column. This can be effected either by means of pipe connection of the stripper column or direct mounting of the stripper column below the absorber column. This direct coupling can be effected since the pressure in the stripping column section and absorption column section is the same. Suitable stripping columns are, for example, tray columns having bubble-cap trays, centrifugal trays and/or sieve trays, columns having structured packings, for example sheet metal packings having a specific surface area of 100 to 1000 m²/m³ such as Mellapak® 250 Y, and columns having random packings. However, also useful are trickle towers and spray towers, and also rotary columns, pan scrubbers, cross-spray scrubbers and rotary scrubbers. Suitable gases are, for example, nitrogen or methane.

In one embodiment of the process according to the invention, in step Db), stripping is effected with a methane-comprising gas stream. More particularly, this gas stream (stripping gas) comprises>90% by volume of methane.

The absorbent stream 17 laden with C₄ hydrocarbons can be heated in a heat exchanger and subsequently passed as stream 19 into a desorption column H. In one process variant, the desorption step Db) is performed by decompressing and stripping the laden absorbent by means of a steam stream 23.

The absorbent 20 regenerated in the desorption stage can be cooled in a heat exchanger. The cooled stream 21 comprises, as well as the absorbent, also water which is removed in the phase separator I.

Low boilers present in the process gas stream, for example ethane or propane, and high-boiling components such as benzaldehyde, maleic anhydride and phthalic anhydride, can accumulate in the absorbent circulation stream. In order to limit the accumulation, a purge stream 25 can be drawn off. Alone or combined with streams 3a and/or 9a and/or 13a, this can be separated in a distillation column T (FIG. 2) according to the prior art into low boilers 36, regenerated absorbent 26 and/or regenerated cooling medium 10 and/or 15 (FIGS. 1 and 2) and high boilers 37. Preferably, it is supplied directly to streams 15 and/or 10 and/or 6, in order to wash the C₄ hydrocarbons still dissolved in streams 25 and/or 3a and/or 9d and/or 13a back into the process gas stream. If stream 25 is separated in the distillation column T, streams 36 and 37 can, for example, be incinerated and hence utilized energetically. Of course, streams 13a and/or 9a and/or 3a can also be purified in a distillation column T without stream 25 and be recycled into the process.

The circulation rate of the absorbent stream 21b can be kept constant by addition of unladen solvent 26. The absorbent stream 21b can be recycled into the absorption stage G.

Since the loading of the water stream 21a with secondary components also increases with time, this can be partially vaporized and the circulation rate of the water stream can be kept constant by adding unladen water 24.

The C₄ product gas stream 27 consisting essentially of n-butane, n-butenes and butadiene comprises generally 20 to 80% by volume of butadiene, 0 to 80% by volume of n-butane, 0 to 10% by volume of 1-butene, 0 to 50% by volume of 2-butenes and 0 to 10% by volume of methane, where the total amount adds up to 100% by volume. In addition, small amounts of isobutane may be present.

A portion of the condensed top discharge from the desorption column comprising principally C₄ hydrocarbons is recycled as stream 25 into the top of the column, in order to increase the separation performance of the column.

The liquid (stream 30) or gaseous (stream 29) C₄ product streams leaving the condenser are subsequently separated by extractive distillation in step E) with a butadiene-selective solvent into a stream 31 comprising butadiene and the selective solvent and a stream 32 comprising butanes and n-butenes.

The extractive distillation can be performed, for example, as described in “Erdöl und Kohle-Erdgas-Petrochemie”, volume 34 (8), pages 343 to 346 or “Ullmanns Enzyklopädie der Technischen Chemie”, volume 9, 4th edition 1975, pages 1 to 18. For this purpose, the C₄ product gas stream is contacted in an extraction zone with an extractant, preferably an N-methylpyrrolidone (NMP)/water mixture. The extraction zone is generally configured in the form of a scrubbing column comprising trays, random packings or structured packings as internals.

This generally has 30 to 70 theoretical plates, in order that a sufficiently good separating action is achieved. Preferably, the scrubbing column has a re-scrubbing zone in the top of the column. This re-scrubbing zone serves for recovery of the extractant present in the gas phase with the aid of a liquid hydrocarbon return stream, for which the top fraction is condensed beforehand. The mass ratio of extractant to C₄ product gas stream in the feed of the extraction zone is generally 10:1 to 20:1. The extractive distillation is preferably operated at a bottom temperature in the range from 100 to 250° C., especially at a temperature in the range from 110 to 210° C., a top temperature in the range from 10 to 100° C., especially in the range from 20 to 70° C., and a pressure in the range from 1 to 15 bar, especially in the range from 3 to 8 bar. The extractive distillation column has preferably 5 to 70 theoretical plates.

Suitable extractants are butyrolactone, nitriles such as acetonitrile, propionitrile, methoxypropionitrile, ketones such as acetone, furfural, N-alkyl-substituted lower aliphatic acid amides such as dimethylformamide, diethylformamide, dimethylacetamide, diethylacetamide. N-formylmorpholine, N-alkyl-substituted cyclic acid amides (lactams) such as N-alkylpyrrolidones, especially N-methylpyrrolidone (NMP). In general, alkyl-substituted lower aliphatic acid amides or N-alkyl-substituted cyclic acid amides are used. Particularly advantageous are dimethylformamide, acetonitrile, furfural and especially NMP.

However, it is also possible to use mixtures of these extractants with one another, for example of NMP and acetonitrile, mixtures of these extractants with co-solvents and/or tert-butyl ethers, e.g. methyl tert-butyl ether, ethyl tert-butyl ether, propyl tert-butyl ether, n- or isobutyl tert-butyl ether. NMP is particularly suitable, preferably in aqueous solution, preferably with 0 to 20% by weight of water, more preferably with 7 to 10% by weight of water, especially with 8.3% by weight of water.

The top product stream from the extractive distillation column comprises essentially butane and butenes and small amounts of butadiene and is drawn off in gaseous or liquid form. In general, the stream consisting essentially of n-butane and 2-butene comprises up to 100% by volume of n-butane, 0 to 50% by volume of 2-butene and 0 to 3% by volume of further constituents such as isobutane, isobutene, propane, propene and c₅⁺ hydrocarbons.

The stream consisting essentially of n-butane and 2-butene, with or without methane, can be fed fully or partly, or else not fed, into the C₄ feed of the ODH reactor. Since the butene isomers in this recycle stream consist essentially of 2-butenes, and 2-butenes are generally oxidatively dehydrogenated more slowly to butadiene than 1-butene, this recycle stream can be catalytically isomerized before being fed into the ODH reactor. As a result, it is possible to adjust the isomer distribution in accordance with the isomer distribution present at thermodynamic equilibrium. In addition, the stream can also be fed to a further workup in order to separate butanes and butenes from one another and to recycle the butenes fully or partly into the oxydehydrogenation. The stream can also go into maleic anhydride production.

In a step F), the stream comprising butadiene and the selective solvent is distillatively separated into a stream consisting essentially of the selective solvent and a stream comprising butadiene.

The stream obtained at the bottom of the extractive distillation column generally comprises the extractant, water, butadiene and small proportions of butenes and butane and is fed to a distillation column. Butadiene can be obtained therein overhead or as a side draw. At the bottom of the distillation column, a stream comprising extractant, with or without water, is obtained, the composition of the stream comprising extractant and water corresponding to the composition as added to the extraction. The stream comprising extractant and water is preferably passed back into the extractive distillation.

If the butadiene is obtained via a side draw, the extraction solution thus drawn off is transferred into a desorption zone, and the butadiene is once again desorbed and re-scrubbed out of the extraction solution. The desorption zone may be configured, for example, in the form of a scrubbing column having 2 to 30 and preferably 5 to 20 theoretical plates, and optionally a re-scrubbing zone having, for example, 4 theoretical plates. This re-scrubbing zone serves for recovery of the extractant present in the gas phase with the aid of a liquid hydrocarbon return stream, for which the top fraction is condensed beforehand. As internals, structured packings, trays or random packings are provided. The distillation is performed preferably at a bottom temperature in the range from 100 to 300° C., especially in the range from 150 to 200° C., and a top temperature in the range from 0 to 70° C., especially in the range from 10 to 50° C. The pressure in the distillation column is preferably in the range from 1 to 10 bar. In general, a reduced pressure and/or an elevated temperature exists in the desorption zone compared to the extraction zone.

The product of value stream 34 obtained at the top of the column comprises generally 90 to 100% by volume of butadiene, 0 to 10% by volume of 2-butene and 0 to 10% by volume of n-butane and isobutane. For further purification of the butadiene, a further distillation can be performed in accordance with the prior art.

### EXAMPLES

### Comparative Example

The example describes the normal state of operation with 6% by volume of oxygen at the end of the ODH reactor without addition of methane.

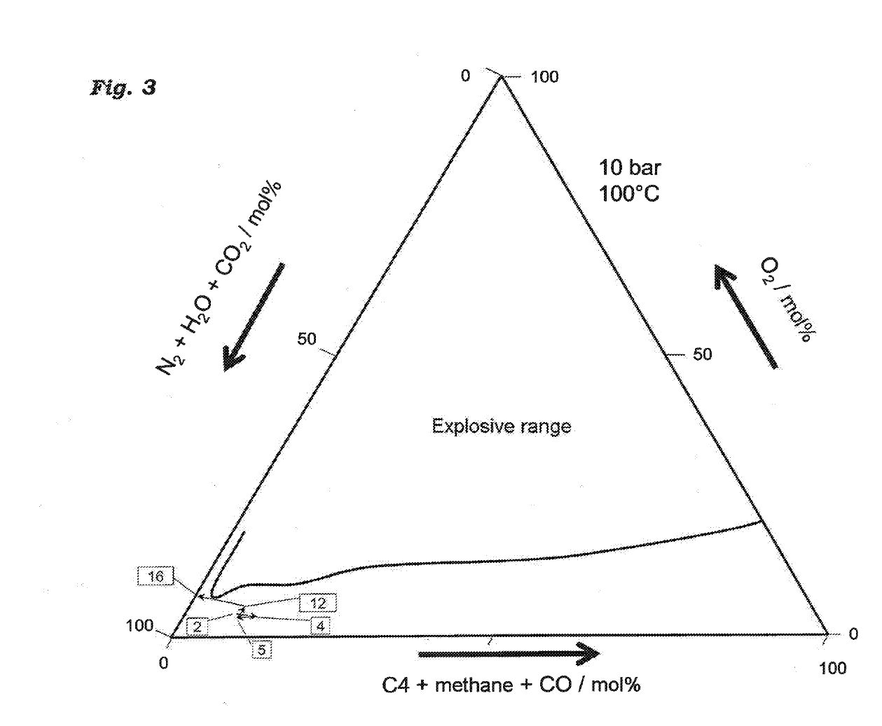

FIG. 3 shows that, under these process conditions, the explosion range range is just touched during the C₄ absorption. The numbers here indicate the composition of streams 2, 4, 5, 12 and 16 in the diagram. In the absorber column, it is possible to observe only a margin of 0.8% by volume of oxygen from the LOC (lowest oxygen concentration, the lowest oxygen concentration at which an explosion can still occur). Thus, in the event of fluctuations in the process, caused, for example, by faults or failures in valves or flowmeters, it is possible that explosive gas mixtures are formed and there is not a quick enough reaction thereto. An explosion thus cannot be completely ruled out, and the oxygen-conducting apparatuses in question thus have to be designed to withstand pressure surges.

TABLE 1
Simulation data for comparative example

| | | Stream number | | | | |
|---|---|---|---|---|---|---|
| | | 2 | 4 | 5 | 12 | 16 |
| Temperature | ° C. | 380 | 88 | 53 | 40 | 35 |
| Pressure | bar | 1.5 | 1.4 | 1.35 | 10.042 | 10 |
| Flow rate | kmol/h | 4739 | 5044 | 4751 | 4213 | 3823 |
| PROPANE | mol % | 0.00 | 0.00 | 0.00 | 0.00 | 0.00 |
| PROPENE | mol % | 0.00 | 0.00 | 0.00 | 0.00 | 0.00 |
| BUTANE | mol % | 1.38 | 1.32 | 1.41 | 1.55 | 0.02 |
| I-BUTANE | mol % | 0.00 | 0.00 | 0.00 | 0.00 | 0.00 |
| I-BUTENE | mol % | 0.12 | 0.12 | 0.13 | 0.14 | 0.00 |
| 1-BUTENE | mol % | 0.00 | 0.00 | 0.00 | 0.00 | 0.00 |
| C-2- | mol % | 0.54 | 0.52 | 0.56 | 0.61 | 0.01 |
| BUTENE | | | | | | |
| T-2- | mol % | 0.76 | 0.72 | 0.77 | 0.85 | 0.01 |
| BUTENE | | | | | | |
| 1,3-BUTA- | mol % | 6.32 | 6.01 | 6.44 | 7.10 | 0.04 |
| DIENE | | | | | | |
| H2O | mol % | 11.73 | 11.07 | 10.58 | 0.73 | 0.52 |
| ETHENE | mol % | 0.02 | 0.02 | 0.02 | 0.02 | 0.02 |
| I-PENTANE | mol % | 0.00 | 0.00 | 0.00 | 0.00 | 0.00 |
| Mesitylene | mol % | 0.03 | 5.92 | 1.20 | 0.06 | 0.06 |
| CO2 | mol % | 1.54 | 1.45 | 1.54 | 1.73 | 1.59 |
| H2 | mol % | 0.01 | 0.01 | 0.01 | 0.01 | 0.01 |
| N2 | mol % | 70.73 | 66.46 | 70.56 | 79.55 | 89.30 |
| O2 | mol % | 6.00 | 5.64 | 5.99 | 6.75 | 7.43 |
| CO | mol % | 0.79 | 0.74 | 0.79 | 0.89 | 0.98 |

### Example 1

The example describes the use of methane in the gas stream in reactor, quench, compressor and absorber column, which ensures that the gas composition is always at least 2% by volume away from the explosion limit. A portion of the methane can be recycled from the offgas stream of the absorber column into the reactor. The margin of 2% by volume from the explosion limit can achieve the effect that, for industrial purposes, leaving of the operating state in the event of fluctuations in the process, caused by faults or failures in valves or flowmeters, can be detected sufficiently rapidly and hence reacted to sufficiently rapidly to avoid entering of the explosion range. Useful technical solutions are to switch off the oxygen supply on attainment of the limit, inertization, to decouple the apparatuses and to switch the gas stream to a flare.

From the ODH reactor A, a process gas 2 having a temperature of 380° C., a pressure of 1.3 bar and the composition shown in table 2 is provided. This gas stream is cooled down to a temperature of 90° C. in the quench section B with a mesitylene circulation stream having a temperature of 35° C. and. This removes a number of the secondary components from the gas stream and the composition of the process gas stream is altered to the concentrations shown for stream 4. The mass ratio of the circulation stream 3b to the process gas 2 and to the purge stream 3a is 1:1:0.067.

The gas stream 4 is cooled further to 45° C. in the second quench stage C with a further mesitylene circulation stream 9b which enters the quench inlet at the top end at 35° C. The resulting gas stream 5 has the composition shown in table 2, while the mass ratio between stream 4 and 9b is 1:2.38, and a purge stream 6 having a proportion of 1% is drawn off from stream 9 and passed into the first quench section.

The gas stream 5 is compressed to 10 bar absolute in a 4-stage compressor having intermediate coolers as shown in FIG. 1. The purge streams 13a, 13a′, 13a″ and 13a′″ are all fed to stream 10.

The resulting gas stream 12′″ (output stream of the heat exchanger F′″ beyond the 4th compression stage) has a temperature of 40° C. and the composition shown in table 2. This stream is separated in the absorber column G by the absorbent stream 21b which is conducted in countercurrent and enters the column at 10 bar absolute and 35° C. at the top of the column into a gas stream 16 and an absorbent stream laden principally with C₄ hydrocarbons. The laden absorbent stream is freed of oxygen by a stream 18 consisting of nitrogen having a temperature of 35° C. to such an extent that the gas stream 27 leaving the desorber column has only 20 ppm of oxygen. The mass ratio between streams 12′″ and 21 b is 1:4.6 and the mass ratio between 21b and 18 is 1:0.003.

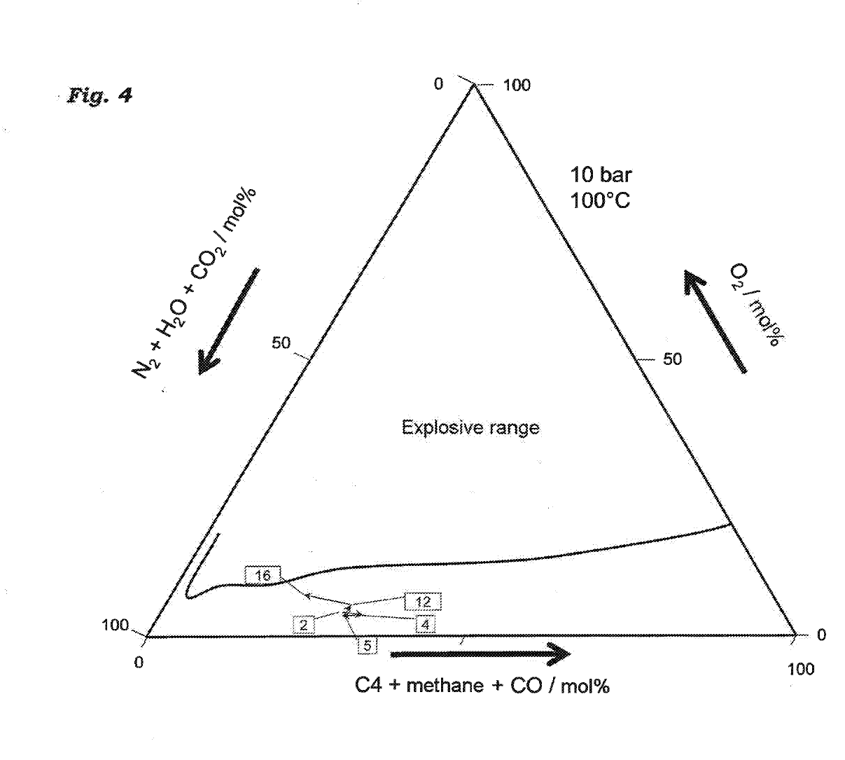

FIG. 4 shows that, by virtue of the higher overall proportion by volume, generated by the methane stream, of organic compounds in the gas stream in the absorber column, a greater margin can be maintained from the explosive range. The numbers indicate the composition of streams 2, 4, 5, 12 and 16 in the diagram.

TABLE 2
Simulation data for example 1

| | | Stream number | | | | |
|---|---|---|---|---|---|---|
| | | 2 | 4 | 5 | 12 | 16 |
| Temperature | ° C. | 380 | 88 | 45 | 40 | 35.37 |
| Pressure | bar | 1.5 | 1.4 | 1.45 | 10.042 | 10 |
| Flow rate | kmol/h | 4740 | 5045 | 1001 | 4212 | 3772 |
| Concen- | | | | | | |
| trations | | | | | | |
| PROPANE | mol % | 0.00 | 0.00 | 0.00 | 0.00 | 0.00 |
| PROPENE | mol % | 0.00 | 0.00 | 0.00 | 0.00 | 0.00 |
| BUTANE | mol % | 1.38 | 1.32 | 1.41 | 1.55 | 0.02 |
| I-BUTANE | mol % | 0.00 | 0.00 | 0.00 | 0.00 | 0.00 |
| I-BUTENE | mol % | 0.12 | 0.12 | 0.13 | 0.14 | 0.00 |
| 1-BUTENE | mol % | 0.00 | 0.00 | 0.00 | 0.00 | 0.00 |
| C-2- | mol % | 0.54 | 0.52 | 0.56 | 0.61 | 0.01 |
| BUTENE | | | | | | |
| T-2- | mol % | 0.76 | 0.72 | 0.77 | 0.85 | 0.01 |
| BUTENE | | | | | | |
| 1,3-BUTA- | mol % | 6.32 | 6.01 | 6.44 | 7.11 | 0.04 |
| DIENE | | | | | | |
| H2O | mol % | 11.73 | 11.07 | 10.57 | 0.72 | 0.51 |
| ETHENE | mol % | 0.02 | 0.02 | 0.02 | 0.02 | 0.02 |
| I-PENTANE | mol % | 0.00 | 0.00 | 0.00 | 0.00 | 0.00 |
| Mesitylene | mol % | 0.03 | 5.92 | 1.20 | 0.06 | 0.06 |
| CO2 | mol % | 1.53 | 1.43 | 1.52 | 1.71 | 1.70 |
| H2 | mol % | 0.01 | 0.01 | 0.01 | 0.01 | 0.01 |
| N2 | mol % | 53.05 | 49.85 | 52.92 | 59.68 | 67.47 |
| O2 | mol % | 6.00 | 5.64 | 5.99 | 6.75 | 7.54 |
| CH4 | mol % | 17.80 | 16.73 | 17.76 | 20.00 | 21.73 |
| CO | mol % | 0.69 | 0.65 | 0.69 | 0.78 | 0.87 |

The invention claimed is:

1.-12. (canceled)

13. A process for preparing butadiene from n-butenes, comprising:
A) providing an input gas stream a comprising n-butenes;
B) feeding the input gas stream a comprising n-butenes and an oxygenous gas into at least one oxidative dehydrogenation zone and oxidatively dehydrogenating n-butenes to butadiene, giving a product gas stream b comprising butadiene, unconverted n-butenes, water vapor, oxygen, low-boiling hydrocarbons and high-boiling secondary components, with or without carbon oxides and with or without inert gases;
Ca) cooling the product gas stream b by contacting it with a coolant and condensing at least a portion of the high-boiling secondary components;
Cb) compressing the remaining product gas stream b in at least one compression stage, giving at least one aqueous condensate stream c1 and one gas stream c2 comprising butadiene, n-butenes, water vapor, oxygen and low-boiling hydrocarbons, with or without carbon oxides and with or without inert gases;
Da) removing uncondensable and low-boiling gas constituents comprising oxygen and low-boiling hydrocarbons, with or without carbon oxides and with or without inert gases, as gas stream d2 from the gas stream c2 by absorbing the C₄ hydrocarbons comprising butadiene and n-butenes in an absorbent, giving an absorbent stream laden with C₄ hydrocarbons and the gas stream d2, and
Db) subsequently desorbing the C₄ hydrocarbons from the laden absorbent stream, giving a C₄ product gas stream d1,
which comprises additionally feeding in a methane-comprising gas stream at at least one point in the process section comprising steps B), Ca), Cb) and Da) in such amounts that the formation of an explosive gas mixture in step Da) is avoided.

14. The process according to claim 13, further comprising:
E) separating the C₄ product stream d1 by extractive distillation with a butadiene-selective solvent into a stream e1 comprising butadiene and the selective solvent and a stream e2 comprising n-butenes;
F) distilling the stream f2 comprising butadiene and the selective solvent into a stream g1 consisting essentially of the selective solvent and a stream g2 comprising butadiene.

15. The process according to claim 13, wherein the methane-comprising gas stream is fed into step B).

16. The process according to claim 13, wherein the methane-comprising gas stream is fed into step Da).

17. The process according to claim 13, wherein the methane content of the gas stream d2 obtained in step Da) is at least 10% by volume.

18. The process according to claim 13, wherein the methane content of the gas stream d2 obtained in step Da) is at least 20% by volume.

19. The process according to claim 13, wherein the nitrogen content of the gas stream d2 obtained in step Da) is at most 10% by volume.

20. The process according to claim 13, wherein the oxygenous gas fed into step B) comprises at least 90% by volume of oxygen.

21. The process according to claim 13, wherein the gas stream d2 removed in step Da) is recycled at least partly into step B).

22. The process according to claim 13, wherein step Da) comprises steps Da1), Da2) and Db):
Da1) absorbing the C₄ hydrocarbons comprising butadiene and n-butenes in a high-boiling absorbent, giving an absorbent stream laden with C₄ hydrocarbons and the gas stream d2,
Da2) removing oxygen from the absorbent stream laden with C₄ hydrocarbons from step Da) by stripping with an uncondensable gas stream, and
Db) desorbing the C₄ hydrocarbons from the laden absorbent stream, giving a C₄ product gas stream d1 comprising less than 100 ppm of oxygen.

23. The process according to claim 22, wherein stripping is effected in step Da2) with a methane-comprising gas stream.

24. The process according to claim 13, wherein the absorbent used in step Da) is an aromatic hydrocarbon solvent.
